(19) United States
(12) Patent Application Publication
(10) Pub. No.: US 2020/0216396 A1
(43) Pub. Date: Jul. 9, 2020

(54) DEUTERATED FORMS OF ISOQUINOLINONE AND METHODS OF USE THEREOF

(71) Applicant: Sojournix, Inc., Waltham, MA (US)

(72) Inventors: David J. Turnquist, Arlington, MA (US); Jayachandra P. Reddy, West Palm Beach, FL (US); Philip B. Graham, Boston, MA (US)

(21) Appl. No.: 16/734,102

(22) Filed: Jan. 3, 2020

Related U.S. Application Data
(60) Provisional application No. 62/817,963, filed on Mar. 13, 2019.
(60) Provisional application No. 62/788,128, filed on Jan. 3, 2019.

(51) Int. Cl. C07D 217/26; A61K 9/70

(52) U.S. Cl. CPC C07D 217/26; A61K 9/7023

(57) ABSTRACT

Disclosed herein are deuterated forms of 2-ethylamino-8-fluoro-3-methyl-1-oxo-1,2-dihydro-isoquinoline-4-carboxylic acid ((S)-cyclopropyl-phenyl-methyl)-amide (also referred to herein as SJB-01) and pharmaceutical compositions comprising such compounds.

### CROSS-REFERENCE TO RELATED APPLICATIONS

This application claims priority to U.S. Provisional Application No. 62/817,963, filed on Mar. 13, 2019, and U.S. Provisional Application No. 62/788,128, filed on Jan. 3, 2019. The entire teachings of the above applications are incorporated herein by reference.

### BACKGROUND OF THE INVENTION

Neurokinin receptors have been suggested as targets for CNS diseases (Albert, Expert Opin. Ther. Patents, 14, 1421-1433, 2004). Neurokinins (or tachykinins) are a family of neuropeptides which include substance P (SP), neurokinin A (NKA), and neurokinin B (NKB). The biological effects of these substances are primarily effected through binding to and activation of the three neurokinin receptors NK1, NK2, and NK3. Although some cross reactivity probably exists, SP has the highest affinity and is believed to be the endogenous ligand for NK1. Similarly, NKA is believed to be the endogenous ligand for NK2, and NKB is believed to be the endogenous ligand for NK3.

NK3 is primarily expressed centrally in regions including cortical regions, such as frontal, parietal and cingulated cortex; nuclei of the amygdale, such as the basal, central and lateral nuclei; the hippocampus; and mesencephalon structures, such as ventral tegmental area, substantia nigra pars compacta, and dorsal raphe nuclei (Spooren et al, Nature Reviews, 4, 967-975, 2005). In addition, morphological studies in rats have provided evidence for putative interactions between NKB neurons and the hypothalamic reproductive axis (Krajewski, J. Comp. Neurol., 489, 372-386, 2005). NKB expression is shown to co-localize with estrogen receptor a and dynorphin in arcuate nucleus neurons (Burke, J. Comp. Neurol., 498, 712-726, 2006; Goodman, Endocrinology, 145, 2959-296, 2004). Further, the NK3 receptor is highly expressed in the hypothalamic arcuate nucleus in neurons, which are involved in the regulation of Gonadotrophin Releasing Hormone (GnRH) release.

Activation of NK-3 receptors has been shown to modulate dopamine, acetylcholine, and serotonin release thereby suggesting a therapeutic utility for NK3 receptor modulators for the treatment of a variety of disorders including psychotic disorders, anxiety, depression, schizophrenia, obesity, pain, and inflammation (Giardina, Expert Opin. Ther. Patents, 10, 939-960, 2000). NK3 receptor modulators may also have therapeutic utility for treating sex hormone-dependent diseases.

New potent and selective antagonists of the NK3 receptor may be of therapeutic value for the preparation of drugs useful in the treatment and/or prevention of a number of diseases or conditions in which NKB and the NK3 receptor are involved. The preparation of these drugs may further include replacing one or more hydrogen atoms with deuterium atoms. Deuterium is a safe, stable, non-radioactive isotope of hydrogen and may form stronger bonds with carbon than hydrogen with carbon. The increased bond strength imparted by deuterium may positively impact various properties of a drug, creating the potential for improved drug efficacy, safety, and/or tolerability. In addition, because the size and shape of deuterium are essentially identical to those of hydrogen, replacement of hydrogen by deuterium would not be expected to affect the biochemical potency and selectivity of the drugs as compared to the original chemical entity that contains only hydrogen.

### SUMMARY OF THE INVENTION

In some aspects, the present disclosure relates to a deuterated form of a compound of formula (I):

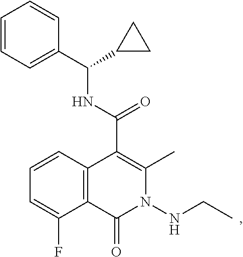 [SMILES: CCNn1c(C)c(C(=O)N[C@H](c2ccccc2)C2CC2)c2cccc(F)c2c1=O]

or a pharmaceutically acceptable salt thereof.

In some embodiments, the compound of formula (I) is deuterated at one or more sites selected from the group consisting of the methyl site, the ethyl site, and the phenyl site.

In some embodiments, the deuterated form of the compound of formula (I) is selected from the group consisting of d5-ethyl-SJB-01, d3-methyl-SJB-01, d3-methyl-d5-phenyl-SJB-01, d5-ethyl-d3-methyl-SJB-01, d5-ethyl-d5-phenyl-SJB-01, d5-phenyl-SJB-01, and d5-ethyl-d3-methyl-d5-phenyl-SJB-01.

In some aspects, the disclosure relates to a compound selected from the

group consisting of:

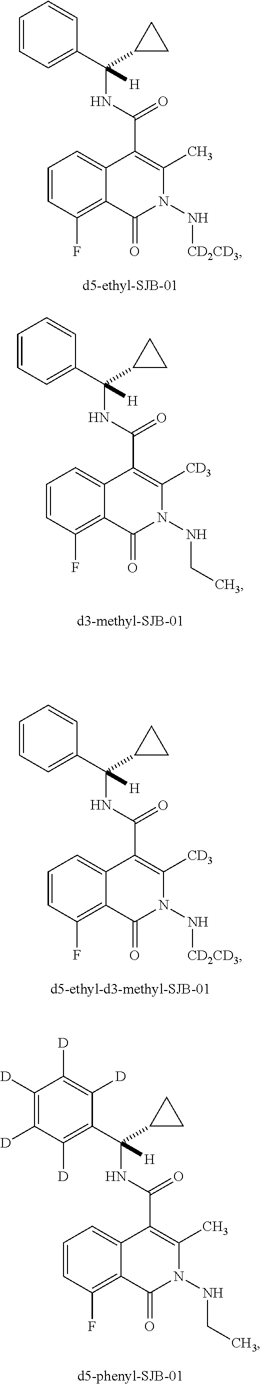

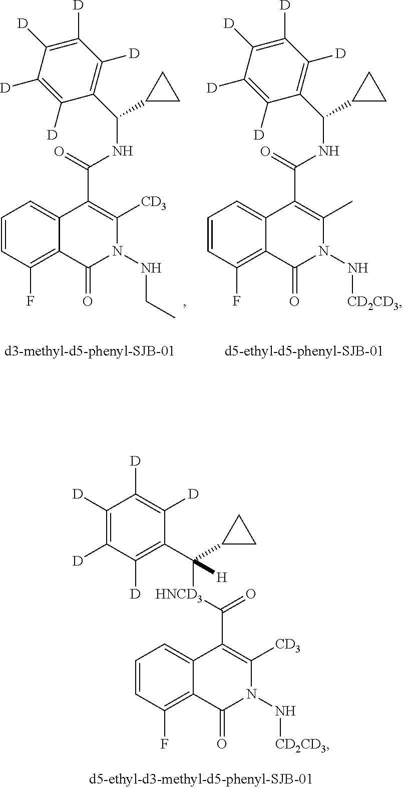

and a pharmaceutically acceptable salt thereof.

In some aspects, the disclosure relates to a compound selected from the group consisting of:

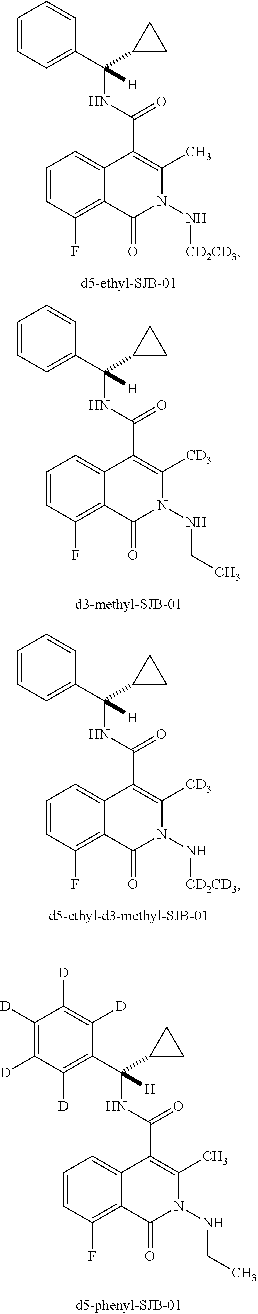

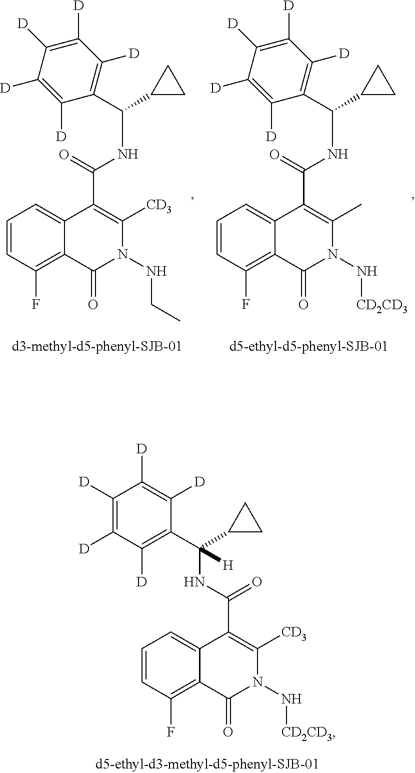

and a pharmaceutically acceptable salt of any one of the foregoing, wherein the compound is substantially pure.

In some aspects, the present disclosure relates to a pharmaceutical composition comprising the compound as described herein and a pharmaceutically acceptable carrier.

In some embodiments, the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

In some embodiments, the pharmaceutical composition is formulated for oral, intraarterial, intravenous or topical administration. In some embodiments, the pharmaceutical composition is formulated as a hard or soft capsule, a tablet, a syrup, a suspension, a solid dispersion, a wafer, or an elixir. In some embodiments, the pharmaceutical composition is formulated as a lotion, a cream, a gel, an oil, an ointment, a salve, or a suspension. In some embodiments, the pharmaceutical composition is formulated as a transdermal patch.

In some embodiments, the pharmaceutical composition described herein further includes an agent that enhances solubility and dispersibility.

In some aspects, the present disclosure relates to a method of treating or preventing a condition or disease in a subject in need thereof, comprising administering to the subject a compound as described herein.

In some aspects, the present disclosure relates to a method of treating or preventing a condition or disease in a subject in need thereof, comprising administering to the subject the pharmaceutical composition as described herein.

In some embodiments, the disease is selected from psychosis; schizophrenia; schizophreniform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; thermal dysregulation in Parkinson's disease; major depression; general anxiety disorder; bipolar disorder (maintenance treatment, recurrence prevention and stabilization); mania; hypomania; cognitive impairment; ADHD; obesity; appetite reduction; cognitive disorders; Alzheimer's disease; Parkinson's disease; thermal dysregulation in Parkinson's disease; pain; convulsions; cough; asthma; airway hyperresponsiveness; microvascular hypersensitivity; bronchoconstriction; chronic obstructive pulmonary disease; urinary incontinence; PTSD; dementia and agitation and delirium in the elderly; inflammatory diseases including irritable bowel syndrome and inflammatory bowel disorders; emesis; pre-eclampsia; airway hyperresponsiveness; reproduction disorders and sex hormone-dependent diseases including but not limited to benign prostatic hyperplasia (BPH), metastatic prostatic carcinoma, testicular cancer, breast cancer, androgen dependent acne, male pattern baldness, endometriosis, abnormal puberty, uterine fibrosis, hormone-dependent cancers, hyperandrogenism, hirsutism, virilization, polycystic ovary syndrome (PCOS), HAIR-AN syndrome (hyperandrogenism, insulin resistance and acanthosis nigricans), ovarian hyperthecosis (HAIR-AN with hyperplasia of luteinized theca cells in ovarian stroma), other manifestations of high intraovarian androgen concentrations (e.g. follicular maturation arrest, atresia, anovulation, dysmenorrhea, dysfunctional uterine bleeding, infertility) and androgen-producing tumor (virilizing ovarian or adrenal tumor); and gynecological disorders and infertility.

In some embodiments, the disease is schizophrenia.

In some embodiments, the disease or condition is excess body fat and/or excess body weight. In some embodiments, the disease or condition is a leptin-related disease. In some embodiments, the disease or condition is a hormonal imbalance. In some embodiments, the disease or condition is associated with non-dipper hypertension. In some embodiments, the disease or condition is associated with post-menopausal hyperandrogenism. In some embodiments, the disease or condition is associated with precocious puberty.

In some embodiments, the condition is hot flashes. In some embodiments, the hot flashes are associated with removal of ovaries or testes of the subject, breast cancer treatment, androgen deprivation therapy, hypogonadism or low serum gonadotropin levels, the subject having leukemia, non-dipper hypertension, carcinoid syndrome, post-menopausal hyperandrogenism, or precocious puberty.

In some aspects, the present disclosure relates to the use of a compound as described herein or a composition as described herein in the manufacture of a medicament for the treatment of a disease.

### BRIEF DESCRIPTION OF THE DRAWINGS

FIG. 1 provides the chemical structure of SJB-01.

FIG. 2 provides the chemical structure of d5-ethyl-SJB-01.

FIG. 3 provides the chemical structure of d3-methyl-SJB-01.

FIG. 4 provides the chemical structure of d5-ethyl-d3-methyl-SJB-01.

FIG. 5 provides the chemical structure of d5-phenyl-SJB-01.

FIG. 6 provides the chemical structure of d5-ethyl-d3-methyl-d5-phenyl-SJB-01.

FIG. 7 provides the chemical structure of d3-methyl-d5-phenyl-SJB-01.

FIG. 8 provides the chemical structure of d5-ethyl-d5-phenyl-SJB-01.

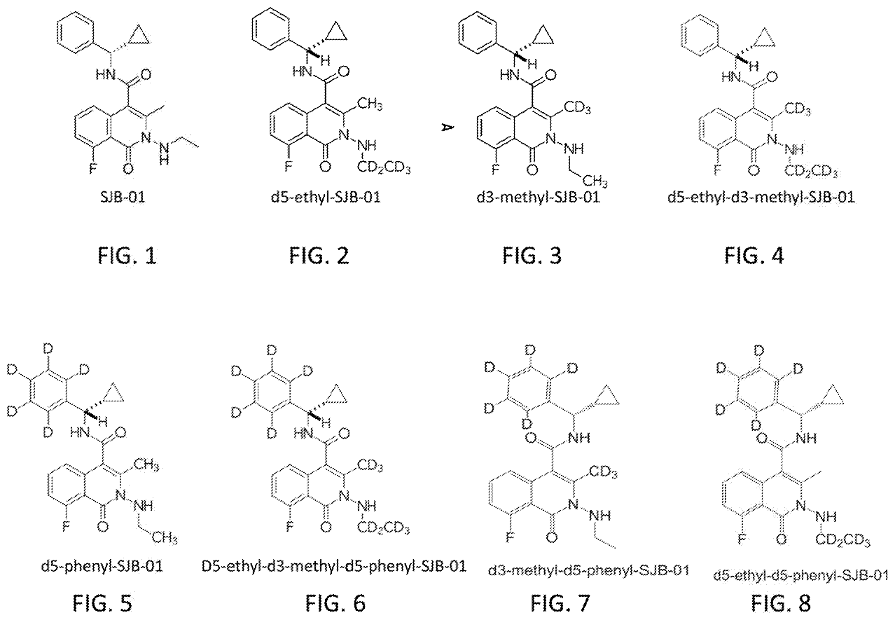

### DETAILED DESCRIPTION OF THE INVENTION

The disclosure relates to the discovery of deuterated forms or analogs of isoquinolinone. In some embodiments the disclosure related to deuterated forms of 2-ethylamino-8-fluoro-3-methyl-1-oxo-1,2-dihydro-isoquinoline-4-carboxylic acid ((S)-cyclopropyl-phenyl-methyl)-amide (also referred to herein as SJB-01), or a pharmaceutically acceptable salt thereof. In some embodiments the disclosure relates to deuterated forms of compounds of formula (I):

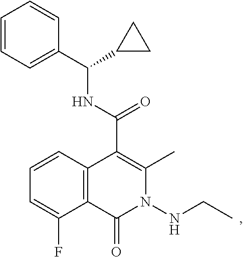 [SMILES: CCNn1c(C)c(C(=O)N[C@H](c2ccccc2)C2CC2)c2cccc(F)c2c1=O]

or a pharmaceutically acceptable salt thereof. Also disclosed are pharmaceutical compositions comprising the described deuterated compounds, methods of treating conditions or diseases, such as psychosis or hot flashes, using compounds described herein or compositions comprising the described compounds, and methods of producing the described compounds.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, kits and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, kits and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, pharmaceutically acceptable salts include, but are not limited to, pharmaceutical acceptable acid addition salts, pharmaceutically acceptable metal salts, ammonium and alkylated ammonium salts. Acid addition salts include salts of inorganic acids as well as organic acids.

Examples of suitable inorganic acids include hydrochloric, hydrobromic, hydroiodic, phosphoric, sulfuric, sulfamic, nitric acids and the like. In certain embodiments the pharmaceutically acceptable salt is a hydrochloride salt.

Examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in J. Pharm. Sci. 1977,66,2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

As used herein, the term “therapeutically effective amount” of a compound means an amount sufficient to cure, alleviate, or partially arrest the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

As used herein, the term “treatment” and “treating” means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relieve the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatments are two separate aspects of the invention. The patient to be treated is preferably a mammal, in particular a human being.

The term “subject” refers to a mammal in need of such treatment, such as a domestic animal (e.g., mouse, rat, guinea pig, dog, cat, household pet, or farm animal), a non-domestic animal (e.g., wildlife), or a human. In some embodiments, the subject is a human.

### Deuterated Compounds

The disclosure relates to deuterated forms of isoquinolinone. In some embodiments the disclosure relates to the deuterated forms of 2-ethylamino-8-fluoro-3-methyl-1-oxo-1,2-dihydro-isoquinoline-4-carboxylic acid ((S)-cyclopropyl-phenyl-methyl)-amide (also referred to herein as SJB-01), or a pharmaceutically acceptable salt thereof. In some aspects the disclosure relates to salts (e.g., pharmaceutically acceptable salts), solvates, hydrates, tautomers, polymorphs, crystalline, or amorphous forms of the deuterated compounds described herein, or a mixture thereof.

2-ethylamino-8-fluoro-3 -methyl-1-oxo-1,2-dihydro-isoquinoline-4-carboxylic acid ((S)-cyclopropyl-phenyl-methyl)-amide has a molecular structure of formula (I):

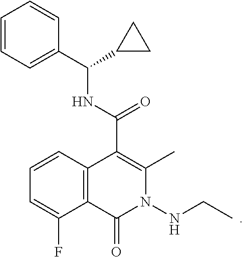 [SMILES: CCNn1c(C)c(C(=O)N[C@H](c2ccccc2)C2CC2)c2cccc(F)c2c1=O]

SJB-01 has a molecular formula of C₂₃H₂₄FN₃O₂ and a molecular weight of 393.46 g/mol. In some embodiments, the compound of SJB-01 is deuterated at one or more sites selected from the group consisting of the methyl site, the ethyl site, and the phenyl site.

In some embodiments the deuterated compound is selected from the group consisting of a d5-ethyl-SJB-01 compound, a d3-methyl-SJB-01 compound, a d5-ethyl-d3-methyl-SJB-01 compound, a d5-phenyl-SJB-01 compound, a d3-methyl-d5-phenyl-SJB-01 compound, a d5-ethyl-d5-phenyl-SJB-01 compound, a d5-ethyl-d3-methyl-d5-phenyl-SJB-01 compound, and a pharmaceutically acceptable salt of any one of the foregoing. In some embodiments, the deuterated compound is substantially pure.

Substitution with heavier isotopes such as deuterium, i.e., ²H or D, provide certain therapeutic advantages resulting from greater metabolic stability, for example, increased in vivo half-life, reduced clearance, reduced dosage requirements, improved safety, or improved efficacy. Deuterated therapeutic agents may provide solutions to address important unmet medical needs (S. L. Harbeson, R. D. Tung, Medchem News, 2014, 2, 8-22). Deuterated forms of 2-ethylamino-8-fluoro-3-methyl-1-oxo-1,2-dihydro-isoquinoline-4-carboxylic acid ((S)-cyclopropyl-phenyl-methyl)-amide exhibit slower metabolization, while retaining the potency, selectivity, and physical properties of the non-deuterated forms of 2-ethylamino-8-fluoro-3-methyl-1-oxo-1,2-dihydro-isoquinoline-4-carboxylic acid ((S)-cyclopropyl-phenyl-methyl)-amide. Any atom in the compounds of the disclosure not specifically labelled as an isotope is meant to represent the given element at about its natural isotopic abundance. For example, H represents protium (1H) with a natural abundance of 99.985% and deuterium (2H) with a natural abundance of 0.015%. While the natural isotopic abundance may vary in a synthesized compound based on the reagents used in the synthesis, the concentration of naturally abundant stable hydrogen isotopes such as deuterium is negligible compared to the concentration of stable isotopic substitution in the compounds of the disclosure.

Thus, when a particular position of the compounds of the disclosure contains a deuterium atom, the concentration of deuterium at that position is substantially greater than the natural abundance of deuterium, which is 0.015%. In some embodiments, a position containing a deuterium atom has a deuterium enrichment or deuterium incorporation or deuterium concentration of at least 1%, of at least 5%, of at least 10%, of at least 15%, of at least 20%, of at least 25%, of at least 30%, of at least 35%, of at least 40%, of at least 45%, of at least 50%, of at least 55%, of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, or of at least 99%. The term “deuterium enrichment” refers to the percentage of incorporation of deuterium at a given position of the compounds of the disclosure in replacement of protium.

In some embodiments the deuterated isoquinolinone is

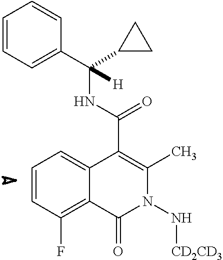

(also referred to herein as d5-ethyl-SJB-01), or a pharmaceutically acceptable salt thereof.

In some embodiments the deuterated isoquinolinone is

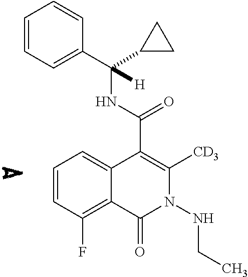 [SMILES: [2H]C([2H])([2H])c1c(C(=O)N[C@H](c2ccccc2)C2CC2)c2cccc(F)c2c(=O)n1NCC]

(also referred to herein as d3-methyl-SJB-01), or a pharmaceutically acceptable salt thereof.

In some embodiments the deuterated isoquinolinone is

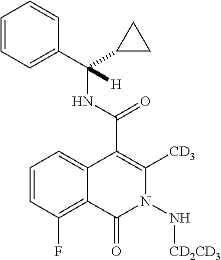

(also referred to herein as d5-ethyl-d3-methyl-SJB-01), or a pharmaceutically acceptable salt thereof.

In some embodiments the deuterated isoquinolinone is

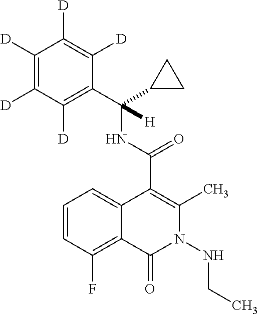 [SMILES: [2H]c1c([2H])c([2H])c([C@@H](NC(=O)c2c(C)n(NCC)c(=O)c3c(F)cccc23)C2CC2)c([2H])c1[2H]]

(also referred to herein as d5-phenyl-SJB-01), or a pharmaceutically acceptable salt thereof.

In some embodiments the deuterated isoquinolinone is

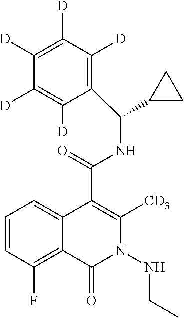 [SMILES: [2H]c1c([2H])c([2H])c([C@@H](NC(=O)c2c(C([2H])([2H])[2H])n(NCC)c(=O)c3c(F)cccc23)C2CC2)c([2H])c1[2H]]

(also referred to herein as d3-methyl-d5-phenyl-SJB-01), or a pharmaceutically acceptable salt thereof.

In some embodiments the deuterated isoquinolinone is

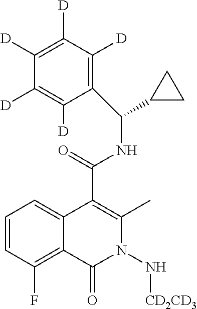

(also referred to herein as d5-ethyl-d5-phenyl-SJB-01), or a pharmaceutically acceptable salt thereof.

In some embodiments the deuterated isoquinolinone is

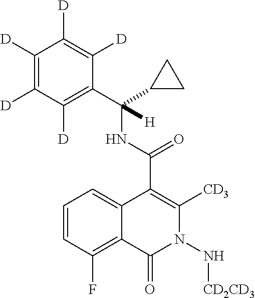

(also referred to herein as d5-ethyl-d3-methyl-d5-phenyl-SJB-01), or a pharmaceutically acceptable salt thereof.

The one or more deuterated compounds of 2-ethylamino-8-fluoro-3-methyl-1-oxo-1,2-dihydro-isoquinoline-4-carboxylic acid ((S)-cyclopropyl-phenyl-ethyl)-amide may be prepared following a similar synthetic route as that of 2-ethylamino-8-fluoro-3 -methyl-1 -oxo-1,2-dihydro-isoquinoline-4-carboxylic acid ((S)-cyclopropyl-phenyl-methyl)-amide.

The synthesis of the compounds described herein can be readily achieved by those of ordinary skill. Such methods can be carried out utilizing corresponding deuterated and optionally, other isotope-containing reagents and/or intermediates to synthesize the compounds described herein, or invoking standard synthetic protocols known in the art for introducing isotopic atoms to a chemical structure, such as those described in in PCT patent publication WO2000/27825; European Patent 1 002 795; U.S. Pat. No. 7,241,458; and Ludovici, D W et al, Biorg Med Chem Lett 2001, 11:2235, each incorporated herein by reference.

### Pharmaceutical Compositions

In some embodiments compositions of this disclosure comprise one or more deuterated forms of formula (I). In certain aspects the compositions comprise at least one, at least two, at least three, at least four, or at least five of the deuterated forms described herein. The composition may be a pharmaceutical composition. In some embodiments, this disclosure provides compositions comprising one or more of d5-ethyl-SJB-01, d3-methyl-SJB-01, d5-ethyl-d3-methyl-SJB-01, d5-phenyl-SJB-01, d3-methyl-d5-phenyl-SJB -01, d5-ethyl-d5-phenyl-SJB -01, d5-ethyl-d3-methyl-d5-phenyl-SJB-01 as described herein.

In some embodiments a pharmaceutical composition comprises a deuterated form of a compound of formula (I) or a pharmaceutically acceptable salt thereof and a pharmaceutically acceptable carrier, diluent, or excipient. The pharmaceutical composition may comprise one or more of the deuterated forms described herein. In some aspects the pharmaceutical composition comprises two of the deuterated forms described herein. In other aspects the pharmaceutical composition comprises three of the deuterated forms described herein. In other aspects the pharmaceutical composition comprises four of the deuterated forms described herein. In other aspects the pharmaceutical composition comprises five of the deuterated forms described herein. In some embodiments the compositions described herein may comprise substantially pure deuterated forms, or may be substantially free of other deuterated forms, and/or impurities.

In some embodiments the term “substantially pure” or “substantially free” with respect to a particular deuterated form of a compound means that the composition comprising the form contains less than 95%, less than 90%, less than 80%, less than 70%, less than 60%, less than 50%, less than 40%, less than 30%, less than 20%, less than 10%, less than 5%, or less than 1% by weight of other substances, including other deuterated forms and/or impurities. In certain embodiments, “substantially pure” or “substantially free of” refers to a substance free of other substances, including other deuterated forms and/or impurities. Impurities may, for example, include by-products or left over reagents from chemical reactions, contaminants, degradation products, other deuterated forms, water, and solvents.

In some aspects, a deuterated form of the compound of formula (I) has a purity of greater than 80%, e.g., greater than 85%, greater than 90%, greater than 92.5%, greater than 95%, greater than 96%, greater than 97%, greater than 98%, greater than 99%, greater than 99.5%, greater than 99.8% (and in certain embodiments of any of the foregoing, less than 100%).

In some aspects a pharmaceutical composition comprises a deuterated form of a compound of formula (I) or a pharmaceutically acceptable salt thereof and a pharmaceutically acceptable carrier, diluent, or excipient, wherein the deuterated compound is d5-ethyl-SJB-01. In some aspects a pharmaceutical composition comprises a deuterated form of a compound of formula (I) or a pharmaceutically acceptable salt thereof and a pharmaceutically acceptable carrier, diluent, or excipient, wherein the deuterated compound is d3-methyl-SJB-01. In some aspects a pharmaceutical composition comprises a deuterated form of a compound of formula (I) or a pharmaceutically acceptable salt thereof and a pharmaceutically acceptable carrier, diluent, or excipient, wherein the deuterated compound is d5-ethyl-d3-methyl-SJB-01. In some aspects a pharmaceutical composition comprises a deuterated form of a compound of formula (I) or a pharmaceutically acceptable salt thereof and a pharmaceutically acceptable carrier, diluent, or excipient, wherein the deuterated compound is d5-phenyl-SJB-01. In some aspects a pharmaceutical composition comprises a deuterated form of a compound of formula (I) or a pharmaceutically acceptable salt thereof and a pharmaceutically acceptable carrier, diluent, or excipient, wherein the deuterated compound is d3-methyl-d5-phenyl-SJB-01. In some aspects a pharmaceutical composition comprises a deuterated form of a compound of formula (I) or a pharmaceutically acceptable salt thereof and a pharmaceutically acceptable carrier, diluent, or excipient, wherein the deuterated compound is d5-ethyl-d5-phenyl-SJB-01. In some aspects a pharmaceutical composition comprises a deuterated form of a compound of formula (I) or a pharmaceutically acceptable salt thereof and a pharmaceutically acceptable carrier, diluent, or excipient, wherein the deuterated compound is d5-ethyl-d3-methyl-d5-phenyl-SJB-01. In some aspects a pharmaceutical composition comprises one or more deuterated forms of a compound of formula (I) or a pharmaceutically acceptable salt thereof and a pharmaceutically acceptable carrier, diluent, or excipient, wherein the deuterated compound is selected from the group consisting of d5-ethyl-SJB-01, d3-methyl-SJB-01, d5-ethyl-d3-methyl-SJB-01, d5-phenyl-SJB-01, d3-methyl-d5-phenyl-SJB-01, d5-ethyl-d5-phenyl-SJB-01, d5-ethyl-d3-methyl-d5-phenyl-SJB-01, a pharmaceutically acceptable salt thereof, and combinations thereof.

In some aspects the pharmaceutical composition further comprises one or more additional therapeutic agents and/or active ingredients. In certain aspects a pharmaceutical composition further comprises an anti-psychotic drug. In certain aspects a pharmaceutical composition further comprises one or more drugs having as a side effect hot flashes. In certain aspects a pharmaceutical composition further comprises low-dose hormone replacement therapy. In some aspects a pharmaceutical composition further comprises one or more selective serotonin reuptake inhibitors. In some aspects a pharmaceutical composition further comprises one or more serotonin and norepinephrine reuptake inhibitors (SNRIs).

The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

A pharmaceutically-acceptable carrier employed in conjunction with the compounds described herein is used at a concentration or amount sufficient to provide a practical size to dosage relationship. The pharmaceutically-acceptable carriers, in total, may, for example, comprise from about 40% to about 99.99999% by weight of the pharmaceutical compositions, e.g., from about 60% to about 99.99%, e.g., from about 80% to about 99/97%, from about 90% to about 99.95%, from about 95% to about 99.9%, or from about 98% to about 99%.

Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

Pharmaceutically acceptable compositions can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof in certain embodiments. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used.

Furthermore it will be appreciated that agents may be modified, e.g., with targeting moieties, moieties that increase their uptake, biological half-life (e.g., pegylation), etc.

### Uses of the Deuterated Compounds and Compositions Thereof

In some embodiments, the compounds (e.g., deuterated forms) described herein exhibit activity as modulators of NK3 receptors. In certain embodiments, the compounds described herein exhibit activity as inhibitors of NK3 receptors. In some embodiments the deuterated forms described herein can be administered (e.g., to a subject) alone as a pure or substantially pure compound. In alternative aspects the deuterated forms are administered in the form of a pharmaceutical composition or formulation.

In some embodiments, the present disclosure relates to the compositions described herein for use in therapy as pharmaceuticals, e.g., in treatment of disorders or conditions associated with NK3 receptors or NKB.

In some aspects disclosed herein are methods of treating or preventing a disease or disorder in a subject by administering a deuterated form of the compound of formula (I) or a pharmaceutically acceptable salt thereof. In some aspects disclosed herein are methods of treating or preventing a disease or disorder in a subject by administering the pharmaceutical composition described herein. In some aspects, a pharmaceutical composition comprises one or more deuterated forms of the compound of formula (I). In some aspects, a therapeutically effective amount of the pharmaceutical composition is administered to the subject. The disorder or disease may be selected from psychosis; schizophrenia; schizophreniform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc.); schizoid personality disorder; schizotypal personality disorder; psychosis or schizophrenia associated with major depression, bipolar disorder, Alzheimer's disease or Parkinson's disease; thermal dysregulation in Parkinson's disease; major depression; general anxiety disorder; bipolar disorder (maintenance treatment, recurrence prevention and stabilization); mania; hypomania; cognitive impairment; ADHD; obesity; appetite reduction; excess body weight; excess body fat; cognitive disorders; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; PTSD; dementia and agitation and delirium in the elderly; inflammatory diseases including irritable bowel syndrome and inflammatory bowel disorders; emesis; pre-eclampsia; airway hyperresponsiveness; reproduction disorders and sex hormone-dependent diseases including but not limited to benign prostatic hyperplasia (BPH), metastatic prostatic carcinoma, testicular cancer, breast cancer, androgen dependent acne, male pattern baldness, endometriosis, abnormal puberty, uterine fibrosis, hormone-dependent cancers, hyperandrogenism, hirsutism, virilization, polycystic ovary syndrome (PCOS), HAIR-AN syndrome (hyperandrogenism, insulin resistance and acanthosis nigricans), ovarian hyperthecosis (HAIR-AN with hyperplasia of luteinized theca cells in ovarian stroma), other manifestations of high intraovarian androgen concentrations (e.g. follicular maturation arrest, atresia, anovulation, dysmenorrhea, dysfunctional uterine bleeding, infertility) and androgen-producing tumor (virilizing ovarian or adrenal tumor); gynecological disorders and infertility.

In some aspects disclosed herein are methods of treating or preventing hot flashes in a subject by administering a deuterated form of the compound of formula (I) or a pharmaceutically acceptable salt thereof. In some aspects the hot flashes occur as a result of menopause, removal of ovaries or tests, treatment for breast cancer, androgen deprivation therapy, hypogonadism and low serum gonadotropin levels, leukemia, non-dipper hypertension, carcinoid syndrome, post-menopausal hyperandrogenism, or precocious puberty in males and females. In some aspects the hot flashes are drug-induced hot flashes.

In some aspects disclosed herein are methods of treating gynecological disorders and infertility in a subject by administering a deuterated form of the compound of formula (I) or a pharmaceutically acceptable salt thereof. In some aspects the methods include suppressing the LH-surge in assisted conception. In some embodiments the compounds are administered to cause male castration and/or to inhibit the sex drive in men.

In some aspects disclosed herein are methods for treating an excess of body fat and/or excess body weight (e.g., treating, preventing, arresting, and/or reducing weight gain) in a subject by administering a deuterated form of the compound of formula (I) or a pharmaceutically acceptable salt thereof. In some aspects the methods of treating an excess of body fat and/or excess body weight in a subject includes decreasing body fat and/or body weight; preventing weight gain and/or ceasing weight gain; decreasing or maintaining plasma triglyceride levels; improving leptin resistance; reducing hyperglycemia and/or decreasing incidence or severity of diabetes; reducing hyperlipidaemia and/or hypertriglyceridemia; decreasing food intake; improving at least one condition associated with weight gain including a cardiovascular disorder, a sleep disorder, a metabolic condition, or a diabetes-related condition; at least partially improving (e.g., terminating or reducing in occurrence) a condition selected from binge eating disorder, night eating syndrome, obsessive eating, compulsive eating, or bulimia; preventing or decreasing abdominal fat accumulation.

In some aspects disclosed herein are methods for treating or preventing a leptin-related disease in a subject by administering a deuterated form of the compound of formula (I) or a pharmaceutically acceptable salt thereof. Examples of leptin-related diseases include metabolic disorders such as diabetes (e.g., Type 1 diabetes), cardiovascular diseases or metabolic syndrome; lipid regulation disorders such as lipodystrophy, including congenital and acquired lipodystrophy, dyslipidemia, nonalcoholic fatty liver disease (NAFLD), nonalcoholic steatohepatitis (NASH) or hyperlipidemia; Congenital Leptin Deficiency (CLD); hypothalamic amenorrhea, such as exercise-induced hypothalamic amenorrhea; Rabson-Mendenhall syndrome; and osteoporosis.

In some aspects disclosed herein are methods for treating a subject suffering from a hormonal imbalance by administering a deuterated form of the compound of formula (I) or a pharmaceutically acceptable salt thereof. Subjects who may be suffering from a hormonal imbalance include women subjected to estrogen-lowering therapies, for example for treatment of breast, cervical, or uterine cancers, or for the treatment of women's health disorders such as endometriosis, uterine fibroids, heavy menstrual bleeding and polycystic ovary syndrome (PCOS); women experiencing natural, age-related decreases in estrogens as occurring during peri-menopause and post-menopause; men subjected to androgen-lowering therapies, such as for treatment of prostate-cancer or benign prostatic hyperplasia (BPH); and men experiencing natural, age-related decreases in circulating testosterone.

In some embodiments the disclosure provides a method for the treatment of schizophrenia, the method comprising the administration of a therapeutically effective amount of a deuterated form of the compound of formula (I) or a pharmaceutically acceptable salt thereof to a subject in need thereof. In particular, said treatment includes the treatment of the positive, negative and/or cognitive symptoms of schizophrenia.

In some embodiments the disclosure provides a method of treating cognitive impairment, the method comprising the administration of a therapeutically effective amount of a deuterated form of the compound of formula (I) or a pharmaceutically acceptable salt thereof to a subject in need thereof. In particular, said cognitive impairment is manifested as a decline in working memory, attention and vigilance, verbal learning and memory, visual learning and memory, reasoning and problem solving e.g. executive function, speed of processing and/or social cognition.

In some embodiments a deuterated form of the compound of formula (I) or a pharmaceutically acceptable salt thereof is administered as part of a combination therapy. In some aspects the combination therapy includes co-administration of the deuterated form with a therapeutic agent. In other aspects the combination therapy includes sequential administration of the deuterated form and the therapeutic agent.

In some aspects a deuterated form of the compound of formula (I) or a pharmaceutically acceptable salt thereof is administered in combination with D2 antagonists. In some aspects a deuterated form of the compound of formula (I) or a pharmaceutically acceptable salt thereof is administered in combination with antagonists/inverse agonists/negative modulators/partial agonists of one or more of the targets dopamine D2 receptor, dopamine D3 receptor, dopamine D4 receptor, phosphodiesterase PDE10, serotonin 5-HT_{1A} receptor, serotonin 5-HT₂A receptor, serotonin 5-HT₆ receptor, adrenergic alpha 2 receptor, cannabinoid type 1 receptor, histamine H3 receptor, cyclooxygenases, sodium channels or glycine transporter GlyT1; or with co-agonists/agonists/positive modulators/partial agonists of one or more of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor.

Such combined administration of a deuterated form of the compound of formula (I) and other anti-psychotic compounds may be sequential or concomitant. Examples of D2 antagonists or partial agonists include haloperidol, chlorpromazine, sulpirid, risperidone, ziprasidone, olanzapine, quetiapine, clozapine and aripiprazole.

In some aspects a deuterated form of the compound of formula (I) or a pharmaceutically acceptable salt thereof is administered in combination with low-dose hormone replacement therapy. In certain aspects the deuterated form increases the efficacy of the low-dose hormone replacement therapy.

In some aspects a deuterated form of the compound of formula (I) or a pharmaceutically acceptable salt thereof is administered in combination with one or more selective serotonin reuptake inhibitors. In some aspects a deuterated form of the compound of formula (I) or a pharmaceutically acceptable salt thereof is administered in combination with one or more serotonin and norepinephrine reuptake inhibitors (SNRIs).

In some aspects a deuterated form of the compound of formula (I) or a pharmaceutically acceptable salt thereof is administered in combination with a therapeutic agent or drug that has as a side effect hot flashes.

In some aspects, a deuterated form of the compound of formula (I) or a pharmaceutically acceptable salt thereof is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 1-1000 mg/day of a compound of the present invention, such as 1-500 mg/day, such as 1-100 mg/day, such as 1-50 mg/day, or 2-20 mg/day.

In one embodiment the present invention relates to the use of a deuterated form of the compound of formula (I) or a pharmaceutically acceptable salt thereof in the manufacture of a medicament for the treatment of a disease. In some aspects, the disease is selected from psychosis; schizophrenia; schizophreniform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; thermal dysregulation in Parkinson's disease; major depression; general anxiety disorder; bipolar disorder (maintenance treatment, recurrence prevention and stabilization); mania; hypomania; cognitive impairment; ADHD; obesity; appetite reduction; cognitive disorders; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; PTSD; dementia and agitation and delirium in the elderly; inflammatory diseases including irritable bowel syndrome and inflammatory bowel disorders; emesis; pre-eclampsia; airway hyperresponsiveness; reproduction disorders and sex hormone-dependent diseases including but not limited to benign prostatic hyperplasia (BPH), metastatic prostatic carcinoma, testicular cancer, breast cancer, androgen dependent acne, male pattern baldness, endometriosis, abnormal puberty, uterine fibrosis, hormone-dependent cancers, hyperandrogenism, hirsutism, virilization, polycystic ovary syndrome (PCOS), HAIR-AN syndrome (hyperandrogenism, insulin resistance and acanthosis nigricans), ovarian hyperthecosis (HAIR-AN with hyperplasia of luteinized theca cells in ovarian stroma), other manifestations of high intraovarian androgen concentrations (e.g. follicular maturation arrest, atresia, anovulation, dysmenorrhea, dysfunctional uterine bleeding, infertility) and androgen-producing tumor (virilizing ovarian or adrenal tumor); gynecological disorders and infertility.

In some embodiments a deuterated form of the compound of formula (I) or a pharmaceutically acceptable salt thereof is used in the manufacture of a medicament for treating or preventing hot flashes. In some aspects the hot flashes occur as a result of menopause, removal of ovaries or tests, treatment for breast cancer, androgen deprivation therapy, hypogonadism and low serum gonadotropin levels, leukemia, non-dipper hypertension, carcinoid syndrome, post-menopausal hyperandrogenism, or precocious puberty in males and females. In some aspects the hot flashes are drug-induced hot flashes.

In some embodiments a deuterated form of the compound of formula (I) is used in the manufacture of a medicament for treating or preventing gynecological disorders and infertility in a subject by administering a deuterated form of the compound of formula (I) or a pharmaceutically acceptable salt thereof.

In some embodiments a deuterated form of the compound of formula (I) or a pharmaceutically acceptable salt thereof is used in the manufacture of a medicament for treating or preventing an excess of body fat and/or excess body weight (e.g., treating, preventing, arresting, and/or reducing weight gain).

In some embodiments a deuterated form of the compound of formula (I) or a pharmaceutically acceptable salt thereof is used in the manufacture of a medicament for treating or preventing a leptin-related disease. Examples of leptin-related diseases include metabolic disorders such as diabetes (e.g., Type 1 diabetes), cardiovascular diseases or metabolic syndrome; lipid regulation disorders such as lipodystrophy, including congenital and acquired lipodystrophy, dyslipidemia, nonalcoholic fatty liver disease (NAFLD), nonalcoholic steatohepatitis (NASH) or hyperlipidemia; Congenital Leptin Deficiency (CLD); hypothalamic amenorrhea, such as exercise-induced hypothalamic amenorrhea; Rabson-Mendenhall syndrome; and osteoporosis.

In some embodiments a deuterated form of the compound of formula (I) is used in the manufacture of a medicament for treating a subject suffering from a hormonal imbalance. Subjects who may be suffering from a hormonal imbalance include women subjected to estrogen-lowering therapies, for example for treatment of breast, cervical, or uterine cancers, or for the treatment of women's health disorders such as endometriosis, uterine fibroids, heavy menstrual bleeding and polycystic ovary syndrome (PCOS); women experiencing natural, age-related decreases in estrogens as occurring during peri-menopause and post-menopause; men subjected to androgen-lowering therapies, such as for treatment of prostate-cancer or benign prostatic hyperplasia (BPH); and men experiencing natural, age-related decreases in circulating testosterone.

In some embodiments a deuterated form of the compound of formula (I) is used in the manufacture of a cosmetic treatment to stimulate the loss of body weight and/or of body fat in a subject. The “cosmetic treatment” is intended to provide an aesthetic/cosmetic effect in subjects, by improving body appearance through stimulating the loss of body weight and/or of body fat (e.g., reduce cellulite). It enables subjects to stabilize weight and to stay thin without localized fat deposits.

In one embodiment the present invention relates to the use of a compound of the present invention in the manufacture of a medicament for the treatment of schizophrenia. In particular, said treatment includes the treatment of the positive, negative and/or cognitive symptoms of schizophrenia.

In some embodiments the present invention relates to the use of a compound as described herein in the manufacture of a medicament to suppress the LH-surge in assisted conception in a patient.

In some embodiments the present invention relates to the use of a compound as described herein in the manufacture of a medicament to treat and/or prevent leptin-related diseases.

In one embodiment, the present invention relates to the use of a compound of the present invention in the manufacture of a medicament for the treatment of cognitive impairment. In particular, said cognitive impairment is manifested as a decline in working memory, attention and vigilance, verbal learning and memory, visual learning and memory, reasoning and problem solving e.g. executive function, speed of processing and/or social cognition.

In one embodiment, the present invention relates to a compound of the present invention for use in the treatment of a disease selected from psychosis; schizophrenia; schizophreniform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; thermal dysregulation in Parkinson's disease; major depression; general anxiety disorder; bipolar disorder (maintenance treatment, recurrence prevention and stabilization); mania; hypomania; cognitive impairment; ADHD; obesity; appetite reduction; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; gut inflammation; inflammatory bowel syndrome; PTSD; dementia and agitation and delirium in the elderly.

In one embodiment, the present invention relates to a compound of the present invention for use in the treatment of schizophrenia. In particular, said treatment includes the treatment of the positive, negative and/or cognitive symptoms of schizophrenia.

In one embodiment, the present invention relates to a compound of the present invention for use in the treatment of cognitive impairment. In particular, said cognitive impairment is manifested as a decline in working memory, attention and vigilance, verbal learning and memory, visual learning and memory, reasoning and problem solving e.g. executive function, speed of processing and/or social cognition.

In one embodiment, the invention relates to a pharmaceutical composition comprising a composition of the present invention together with an anti-psychotic agent. In one embodiment, said anti-psychotic agent is selected from antagonists/inverse agonists/negative modulators/partial agonists of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or from agonists/positive modulators/partial agonists of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor. Particular examples of such anti-psychotics include haloperidol, chlorpromazine, sulpirid, risperidone, ziprasidone, olanzapine, quetiapine, clozapine and aripoprazole.

The deuterated compounds described herein may be administered in pharmaceutically acceptable solutions, which may routinely contain pharmaceutically acceptable concentrations of salt, buffering agents, preservatives, compatible carriers, adjuvants, and optionally other therapeutic ingredients.

The deuterated forms of the compound of formula (I) may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, deuterated forms of the compound of formula (I) may be administered directly to a tissue. Direct tissue administration may be achieved by direct injection. The deuterated forms may be administered once, or alternatively they may be administered in a plurality of administrations. If administered multiple times, the deuterated forms may be administered via different routes. For example, the first (or the first few) administrations may be made directly into the affected tissue while later administrations may be systemic.

For oral administration, compositions can be formulated readily by combining the deuterated forms with pharmaceutically acceptable carriers well known in the art. Such carriers enable the deuterated forms to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, emulsions, and other non-aqueous vehicles. Examples of non-aqueous solvents are propylene glycol, polyethylene glycol, vegetable oils such as olive oil, and injectable organic esters such as ethyl oleate. Additional examples of non-aqueous vehicles for delivery include 10% 2-hydroxypropyl-β-cyclodextrin (HPbetaCD), 25% polyethylene glycol (15)-hydroxystearate (Solutol HS15®), 25% polyoxyl 35 hydrogenated castor oil (Cremophor EL®), 100% macrogol 6 glycerol caprylocaprate (Softigen 767®); 100% polyethylene glycol (PEG 400), 100% Viscoleo, and 90:10 Gelucire® 44/14:PEG 400.

Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include suppositories, sprays, ointments, cremes, gels, inhalants, dermal patches, implants, etc. Transdermal patches may be placed on the skin and used to deliver a specific dose of medication through the skin and into the bloodstream. In some aspects a transdermal patch provides for controlled release of the pharmaceutical composition.

Conveniently, the compounds of the invention are administered in a unit dosage form containing said compounds in an amount of about 0.1 to 500 mg, such as 1 mg, 5 mg, 10 mg, 50 mg 100 mg, 150 mg, 200 mg or 250 mg of a compound of the present invention.

For parenteral routes such as intravenous, intrathecal, intramuscular and similar administration, typically doses are in the order of about half the dose employed for oral administration.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutical acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

### Kits

The deuterated forms described herein can be provided in a kit. The kit includes (a) one or more deuterated forms of the compound of formula (I), e.g., a composition that includes the one or more compounds and (b) informational material. In some embodiments the kit further includes an anti-psychotic drug. In some aspects the anti-psychotic drug is selected from typical anti-psychotics, atypical anti-psychotics, antagonists/inverse agonists/negative modulators/partial agonists of one or more of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or with agonists/positive modulators/partial agonists of one or more of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor.

Particular examples of such anti-psychotics include haloperidol, chlorpromazine, sulpirid, risperidone, ziprasidone, olanzapine, quetiapine, clozapine and aripiprazole.

The informational material can be descriptive, instructional, marketing or other material that relates to the methods described herein and/or the use of the components for the methods described herein. For example, the informational material may describe methods for administering the compounds, and in some aspects the anti-psychotic drugs, to a subject. The informational material can include instructions to administer the compounds described herein in a suitable manner, e.g., in a suitable dose, dosage form, or mode of administration. In some embodiments, the instructions recommend administering an effective amount of a compound. The informational material can include instructions for selecting a suitable subject.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the inhibitor and/or its use in the methods described herein. Of course, the informational material can also be provided in any combination of formats.

In addition to the compounds and, in some aspects the anti-psychotic drug, the kit can include other ingredients, such as a solvent or buffer, a stabilizer or a preservative, and/or an agent for treating a condition or disorder described herein. Alternatively, the other ingredients can be included in the kit, but in different compositions or containers than the compound. In such embodiments, the kit can include instructions for admixing the compound and the other ingredients, or for using the compound with the other ingredients.

The compound described herein can be provided in any form, e.g., liquid, dried or lyophilized form. It is preferred that the glutamine metabolism inhibitor be substantially pure and/or sterile. When the compound is provided in a liquid solution, the liquid solution preferably is an aqueous solution, with a sterile aqueous solution being preferred. When the compound is provided as a dried form, reconstitution generally is by the addition of a suitable solvent. The solvent, e.g., sterile water or buffer, can optionally be provided in the kit.

The kit can include one or more containers for the composition containing the compound and an anti-psychotic drug. In some embodiments, the kit contains separate containers, dividers or compartments for the compound (e.g., in a composition), the anti-psychotic drug, and informational material. For example, the compound and the anti-psychotic drug can each be contained in a bottle, vial, or syringe, and the informational material can be contained in a plastic sleeve or packet. In other embodiments, the separate elements of the kit are contained within a single, undivided container. For example, the compound (e.g., in a composition) and the anti-psychotic drug are contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the compound (e.g., in a composition) and the anti-psychotic drug. For example, the kit includes a plurality of syringes, ampules, foil packets, or blister packs, each containing a single unit dose of the agent. The containers of the kits can be air tight and/or waterproof.

All patents and other publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### Synthesis of d5-Ethyl-SJB-01

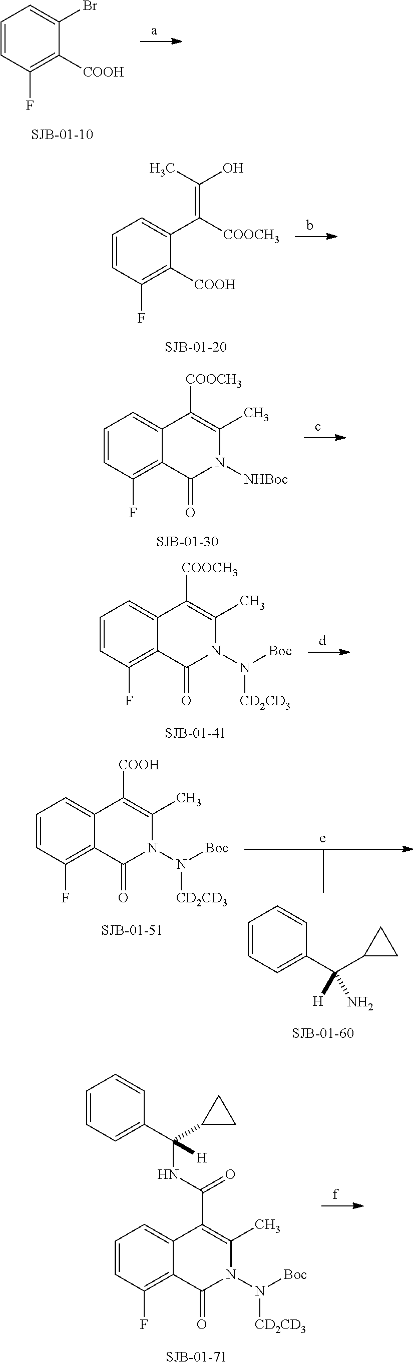

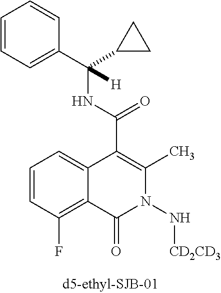

Reagents: a) sodium methyl acetoacetate, cat CuBr, methyl acetoacetate, 50° C., b) Boc-NHNH₂, DCC, HOSu, NMP, 95° C., c) d5-EtBr, K₂CO₃, NMP, 45° C., d) aq NaOH, 45° C., e) SOCl₂, TEA, cat DMF, IPAc, −10 ° C., then SJB-01-60, 25° C., f) HCl in IPAc, IPAc, 0 to 25° C.

### Example 2

### Synthesis of d3-Methyl-SJB-01

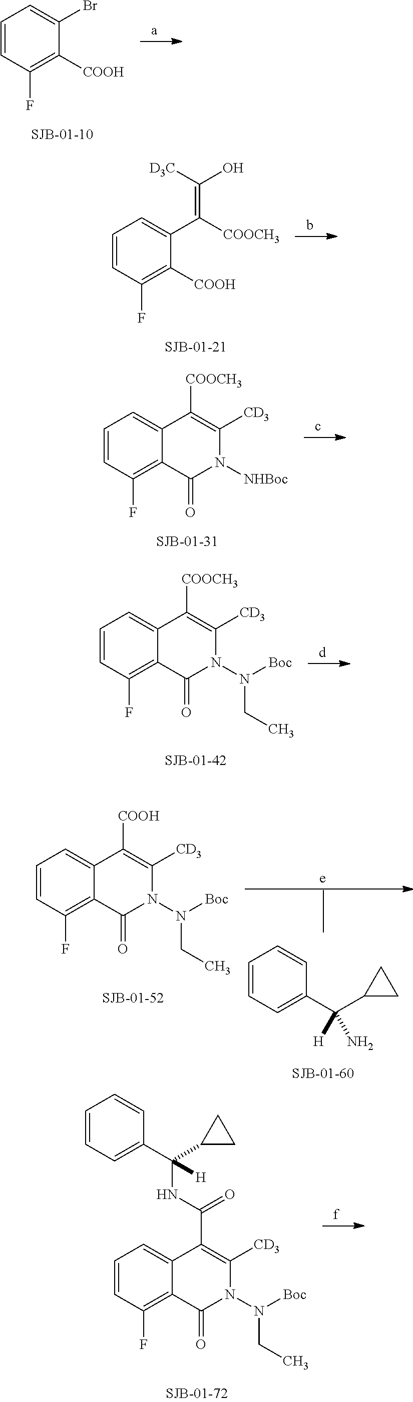

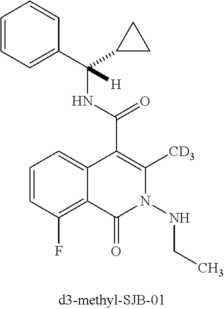 [SMILES: [2H]C([2H])([2H])c1c(C(=O)N[C@H](c2ccccc2)C2CC2)c2cccc(F)c2c(=O)n1NCC]

Reagents: a) sodium d3-methyl acetoacetate, cat CuBr, d3-methyl acetoacetate, 50° C., b) Boc-NHNH₂, DCC, HOSu, NMP, 95° C., c) EtBr, K₂CO₃, NMP, 45° C., d) aq NaOH, 45° C., e) SOCl₂, TEA, cat DMF, IPAc, −10° C., then SJB-01-60, 25° C., f) HCl in IPAc, IPAc, 0 to 25° C.

### Example 3

### Synthesis of d5-Ethyl-d3-Methyl-SJB-01

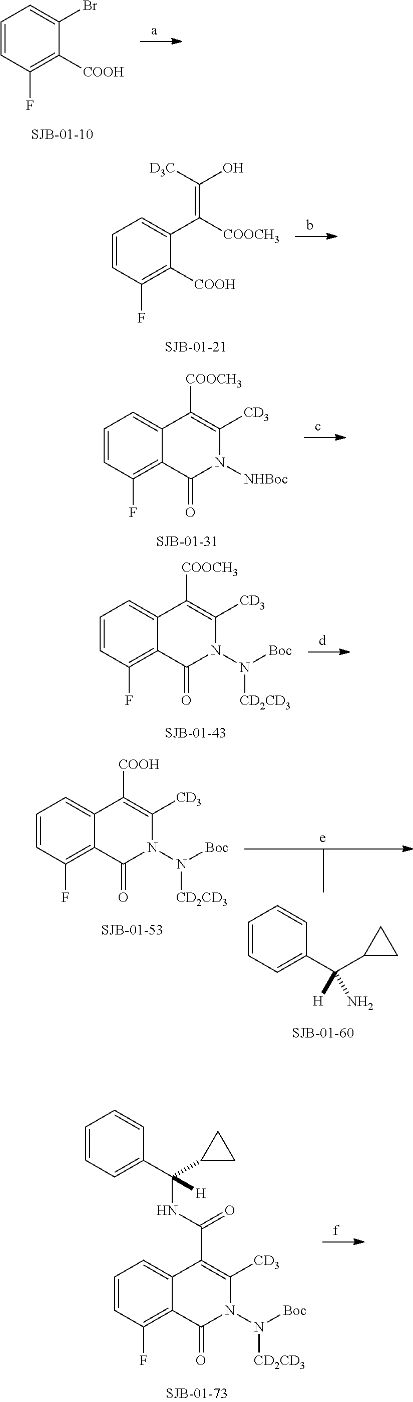

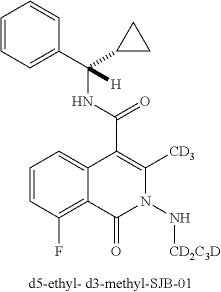

Reagents: a) sodium d3-methyl acetoacetate, cat CuBr, d3-methyl acetoacetate, 50° C., b) Boc-NHNH₂, DCC, HOSu, NMP, 95° C., c) d5-EtBr, K₂CO₃, NMP, 45° C., d) aq NaOH, 45° C., e) SOCl₂, TEA, cat DMF, IPAc, −10° C., then SJB-01-60, 25° C., f) HCl in IPAc, IPAc, 0 to 25° C.

### Example 4

### Synthesis of d5-Phenyl-SJB-01

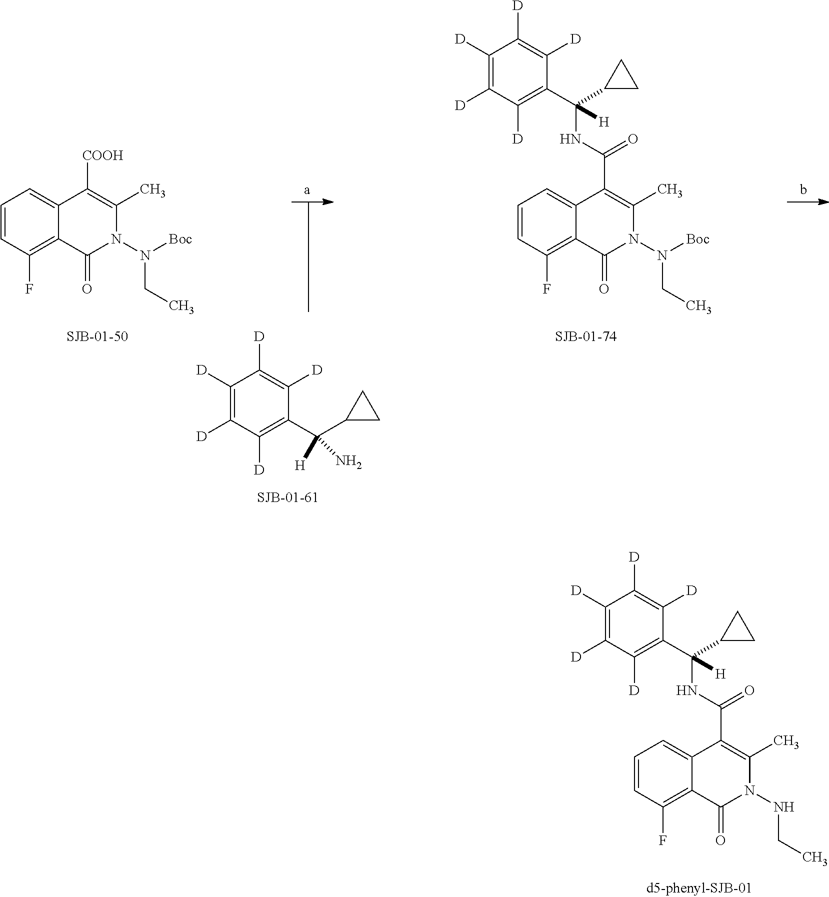 [SMILES: CCN(C(=O)OC(C)(C)C)n1c(C)c(C(=O)O)c2cccc(F)c2c1=O ; [2H]c1c([2H])c([2H])c([C@@H](NC(=O)c2c(C)n(N(CC)C(=O)OC(C)(C)C)c(=O)c3c(F)cccc23)C2CC2)c([2H])c1[2H] ; [2H]c1c([2H])c([2H])c([C@@H](N)C2CC2)c([2H])c1[2H] ; [2H]c1c([2H])c([2H])c([C@@H](NC(=O)c2c(C)n(NCC)c(=O)c3c(F)cccc23)C2CC2)c([2H])c1[2H]]

Reagents: a) SOCl₂, TEA, cat DMF, IPAc, −10 ° C., then SJB-01-61, 25° C., b) HCl in IPAc, IPAc, 0 to 25° C.

### Example 5

### Synthesis of d5-Ethyl-d3-Methyl-d5-Phenyl-SJB-01

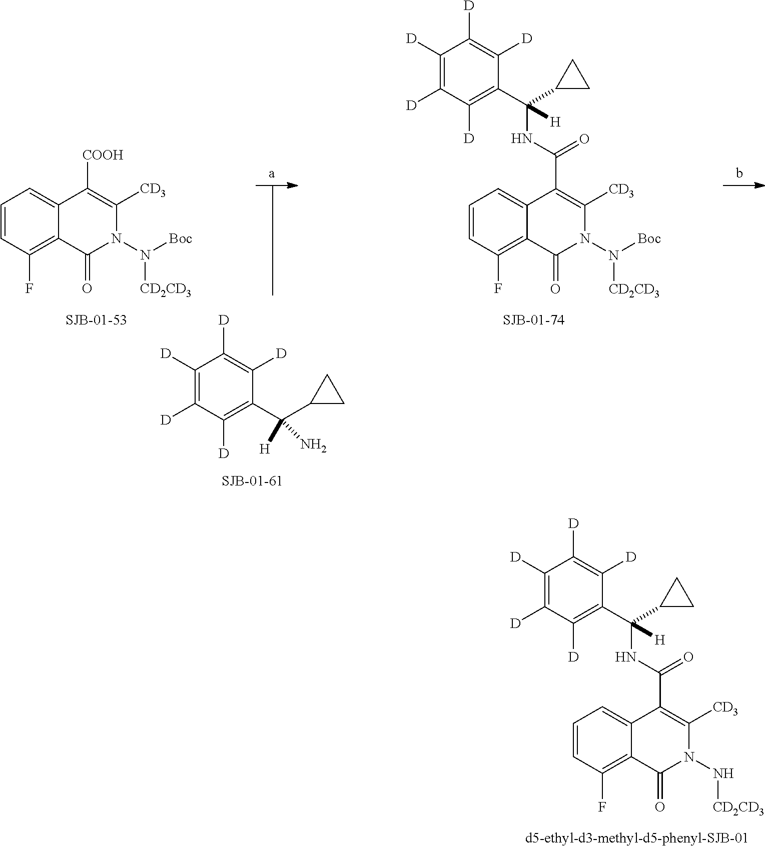

Reagents: a) SOC12, TEA, cat DMF, IPAc, −10° C., then SJB-01-61, 25° C., b) HCl in IPAc, IPAc, 0 to 25° C.

### Example 6

### Synthesis of d3-Methyl-d5-Phenyl-SJB-01

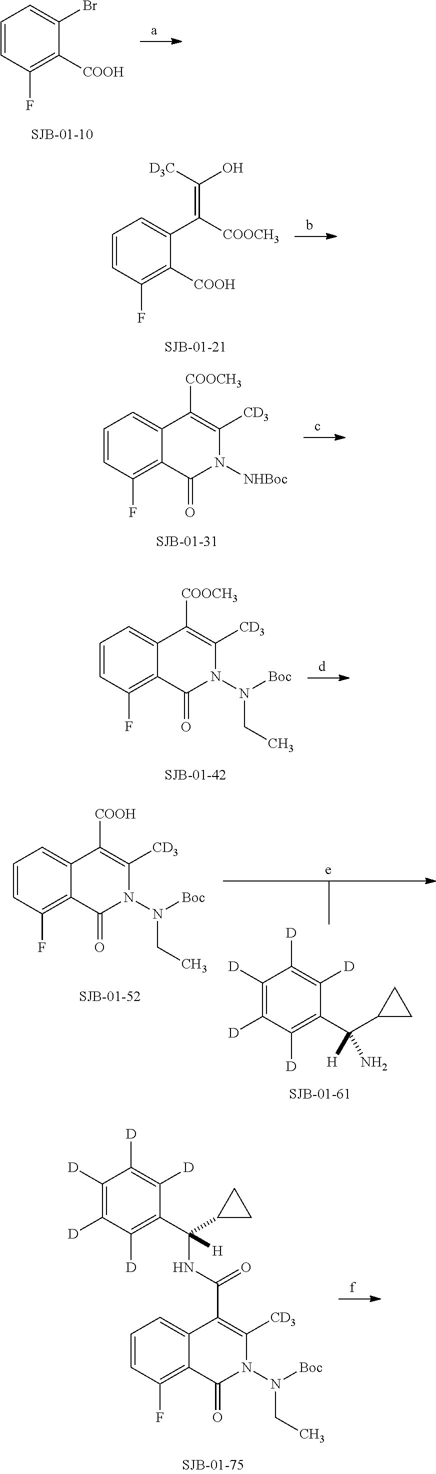

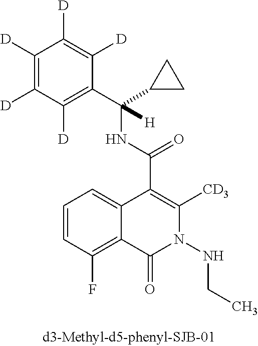 [SMILES: [2H]c1c([2H])c([2H])c([C@@H](NC(=O)c2c(C([2H])([2H])[2H])n(NCC)c(=O)c3c(F)cccc23)C2CC2)c([2H])c1[2H]]

Reagents: a) sodium d3-methyl acetoacetate, cat CuBr, d3-methyl acetoacetate, 50° C., b) Boc-NHNH₂, DCC, HOSu, NMP, 95° C., c) EtBr, K₂CO₃, NMP, 45° C., d) aq NaOH, 45° C., e) SOCl₂, TEA, cat DMF, IPAc, −10° C., then SJX-653-61, 25° C., f) HCl in IPAc, IPAc, 0 to 25° C.

### Example 7

### Synthesis of d5-Ethyl-d5-Phenyl-SJB-01

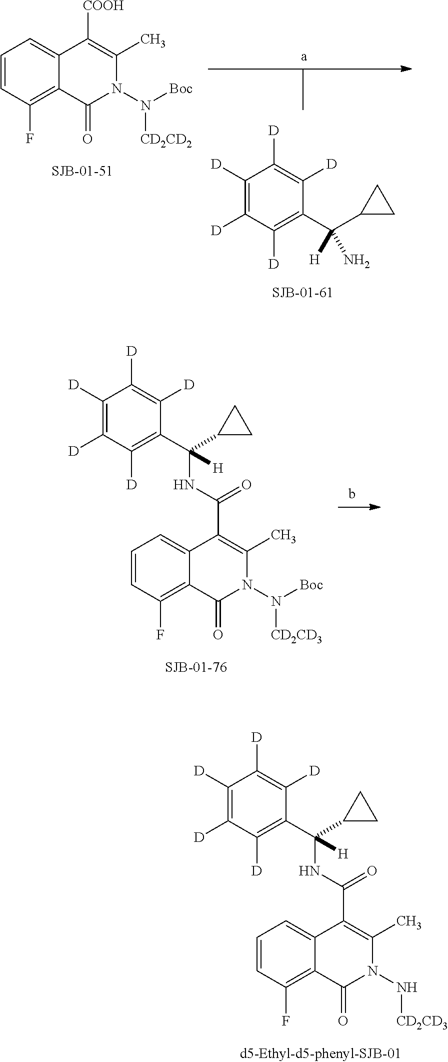

Reagents: a) SOCl₂, TEA, cat DMF, IPAc, −10° C., then SJX-653-61, 25° C., b) HCl in IPAc, IPAc, 0 to 25° C.

### Example 8

### Assessing Properties of Deuterated Forms of SJB-01

One or more compounds of d5-ethyl-SJB-01, d3-methyl-SJB-01, d5-ethyl-d3-methyl-SJB-01, d5-phenyl-SJB-01, d3-methyl-d5-phenyl-SJB-01, d5-ethyl-d5-phenyl-SJB-01, or d5-ethyl-d3-methyl-d5-phenyl-SJB-01 may be assessed using various biological assays known to those of skill in the art.

For example, the in vitro metabolism of a selected compound may be assessed using human and other species, microsomes, and hepatocytes, as well as CYP3A4 supersomes. Measurements would include depletion of the parent, as well as rate of formation of the metabolites. In addition, potency and selectivity for NK3 cab be evaluated through a functional assay (e.g., inhibition of NKB activity), as well as binding assays for NK1 and NK2. The physical properties, solubility, and permeability of the compounds can also be assessed using standard methods known to those of skill in the art. Also, each compound can be evaluated in vivo in an appropriate animal model, or in humans.

What is claimed is:

1. A deuterated form of a compound of formula (I):

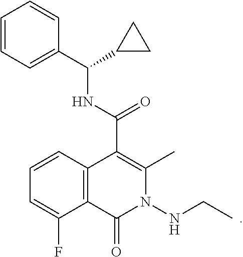 [SMILES: CCNn1c(C)c(C(=O)N[C@H](c2ccccc2)C2CC2)c2cccc(F)c2c1=O]

2. The deuterated form of the compound of claim 1, wherein the deuterated form of the compound is selected from the group consisting of d5-ethyl-SJB-01, d3-methyl-SJB-01, d5-ethyl-d3-methyl-SJB-01, d5-phenyl-SJB-01, d3-methyl-d5-phenyl-SJB-01, d5-ethyl-d5-phenyl-SJB-01, and d5-ethyl-d3-methyl-d5-phenyl-SJB-01.

3. A compound selected from the group consisting of: 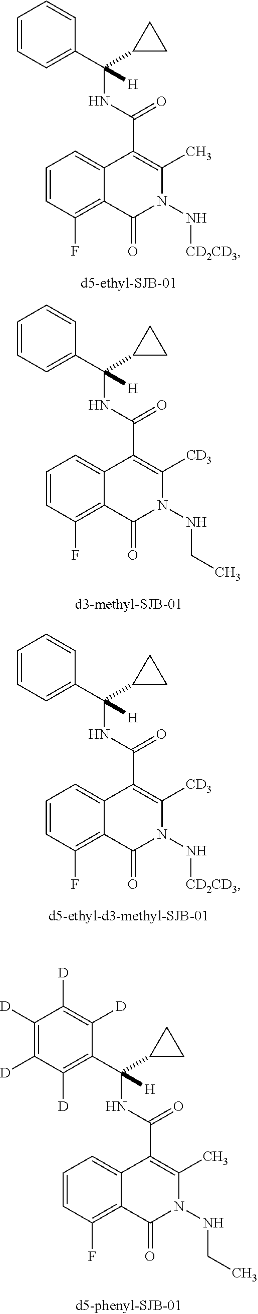 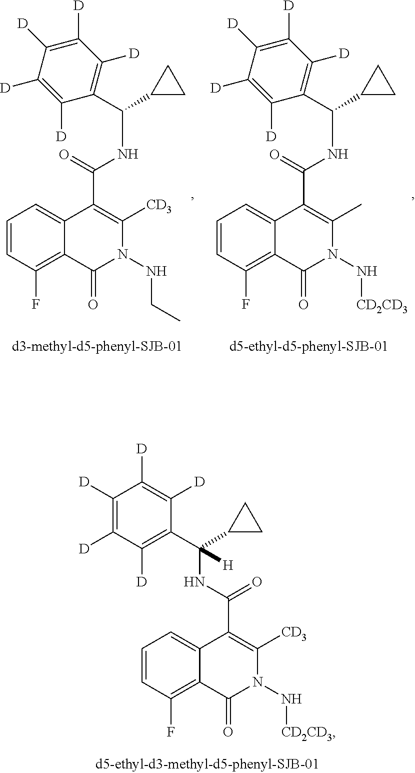
or a pharmaceutically acceptable salt thereof.

4. The compound of claim 3, wherein the compound is substantially pure.

5. A pharmaceutical composition comprising the compound of 1 and a pharmaceutically acceptable carrier.

6. The pharmaceutical composition of claim 5, wherein the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

7. The pharmaceutical composition of claim 5, wherein the pharmaceutical composition is formulated as a hard or soft capsule, a tablet, a syrup, a suspension, a solid dispersion, a wafer, an elixir

8. The pharmaceutical composition of claim 5, wherein the pharmaceutical composition is formulated as a lotion, a cream, a gel, an oil, an ointment, a salve, or a suspension.

9. The pharmaceutical composition of claim 5, wherein the pharmaceutical composition is formulated as a transdermal patch.

10. The pharmaceutical composition of claim 5, further comprising an agent that enhances solubility and dispersibility.

11. A method of treating a subject in need thereof, comprising administering to the subject a compound of claim 1.

12. A method of treating or preventing a condition or disease in a subject in need thereof, comprising administering to the subject the pharmaceutical composition of any one of claims 5.

13. The method of claim 12, wherein the disease is selected from psychosis; schizophrenia; schizophreniform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; thermal dysregulation in Parkinson's disease; major depression; general anxiety disorder; bipolar disorder (maintenance treatment, recurrence prevention and stabilization); mania; hypomania; cognitive impairment; ADHD; obesity; appetite reduction; cognitive disorders; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; PTSD; dementia and agitation and delirium in the elderly; inflammatory diseases including irritable bowel syndrome and inflammatory bowel disorders; emesis; pre-eclampsia; airway hyperresponsiveness; reproduction disorders and sex hormone-dependent diseases including but not limited to benign prostatic hyperplasia (BPH), metastatic prostatic carcinoma, testicular cancer, breast cancer, androgen dependent acne, male pattern baldness, endometriosis, abnormal puberty, uterine fibrosis, hormone-dependent cancers, hyperandrogenism, hirsutism, virilization, polycystic ovary syndrome (PCOS), HAIR-AN syndrome (hyperandrogenism, insulin resistance and acanthosis nigricans), ovarian hyperthecosis (HAIR-AN with hyperplasia of luteinized theca cells in ovarian stroma), other manifestations of high intraovarian androgen concentrations (e.g. follicular maturation arrest, atresia, anovulation, dysmenorrhea, dysfunctional uterine bleeding, infertility) and androgen-producing tumor (virilizing ovarian or adrenal tumor); and gynecological disorders and infertility.

14. The method of claim 12, wherein the disease or condition is associated with non-dipper hypertension, post-menopausal hyperandrogenism, or precocious puberty.

15. The method of claim 12, wherein the condition is hot flashes.

16. The method of claim 15, wherein the hot flashes are associated with one or more of: removal of ovaries or testes of the subject, breast cancer treatment, androgen deprivation therapy, hypogonadism or low serum gonadotropin levels, the subject having leukemia, or carcinoid syndrome.

17. The method of claim 12, wherein the disease or condition is excess body fat and/or excess body weight.

18. The method of claim 12, wherein the disease or condition is a leptin-related disease.

19. The method of claim 12, wherein the disease or condition is a hormonal imbalance.

20. A method of manufacturing a medicament for the treatment of a disease comprising using the compound of claim 1.
